(19) United States
(12) Patent Application Publication
(10) Pub. No.: US 2007/0156014 A1
(43) Pub. Date: Jul. 5, 2007

(54) Angled male member enlargement pump

(76) Inventor: Nikos Zafirakis, Candia, NH (US)

Correspondence Address: Russ Weinzimmer, 614 Nashua St. #204, Milford, NH, 03055, US

(21) Appl. No.: 11/321,107

(22) Filed: Dec. 29, 2005

(51) Int. Cl. A61F 5/00

(52) U.S. Cl. 600/41

(57) ABSTRACT

An angled male member enlargement pump is disclosed, the pump including a transparent enlargement receptacle for receiving a male member to be enlarged; a unidirectional air flow valve for permitting air flow in only one direction, a pump chamber for pulling air along a longitudinal direction of the pump chamber; and an angled pump chamber for establishing and maintaining a substantially 90 degree angle between the one direction through the air flow valve and the longitudinal direction of the pump chamber. The enlargement receptacle includes a pressure relief assembly for allowing the enlargement receptacle to reach ambient pressure. To reduce pressure within the enlargement receptacle, a user moves the T-shaped handle so as to draw air through the unidirectional air flow valve, thereby removing air from the transparent enlargement receptacle. The angled pump chamber allows convenient and comfortable pumping motion while a male member is held within the enlargement receptacle.

### FIELD OF THE INVENTION

The invention generally relates to apparatus for soft tissue enlargement, and specifically to apparatus for enlarging a male member and/or establishing an erection.

### BACKGROUND OF THE INVENTION

Erectile dysfunction (ED) is a known medical condition for which there are many commercially available medications designed to enhance penile blood flow and engorgement. However, the medications that treat ED have side effects. While these medications provide temporary improvement in a patient's ability to establish and maintain an erection, there is a need for external methods for males with erectile dysfunction to establish an erection, and further to enhance penile size, without the risks of side effects of medications, and with a longer lasting benefit.

For males without erectile dysfunction, there nevertheless exists a need to provide a safe means of enlarging the penis for improved erectile extent and quality, as well as enhanced sexual satisfaction during intercourse.

With the growth of and competition within the adult entertainment industry, there is also a need for male entertainers to display increasingly large erections, and therefore there exists a need for male entertainers to have available a safe, easy-to-use means of achieving such erections on a regular basis.

Devices known in the art to assist in the establishment and enhancement of an erection in a male are overly complicated, often having multiple valves, pressure gauges, and spring mechanisms. Some prior art devices require an unspecified source of vacuum, or use electric pumping motors, or incorporate mechanisms that restrict the flow of blood out of the erect penis, or are uncomfortable to use.

### SUMMARY OF THE INVENTION

The angled male member enlargement pump of the invention is an elegantly constructed, easy-to-use, manually-operated penis enlargement pump with a unified assembly providing indirect pneumatic pumping force to a male member, and a convenient and comfortable angled configuration that is especially suitable for solo use. The pump of the invention can also be used with a partner.

The basic methodology applied to enlarge the penis (evacuating air from a chamber around the penis, thereby creating a partial vacuum into which the penis tissue expands) has been documented in the prior art. One feature of the invention is the inclusion of a T-shaped handle for manually operating a pumping mechanism for creating the partial vacuum. Another feature of the invention is the inclusion of a substantially right-angled bend in the structure of the pump of the invention, enabling a user to pump in a direction perpendicular to the direction of longitudinal enlargement of the male member. While providing a unified and rigid assembly, the angle allows the pumping force to be more conveniently and comfortably applied, while also allowing the natural angle of the erection.

The pump of the invention provides a simple, easy-to-manufacture, and easy-to-operate apparatus for purposes of establishing an erection, and with repeated use, for enlarging a penis. The invention includes a substantially cylindrical enlargement receptacle open at one end for inserting a penis; a unidirectional air flow valve cooperative therewith that allows air to be evacuated from the enlargement receptacle without it rushing back into the enlargement receptacle; an angled section that connects the enlargement receptacle to a pump chamber, the substantially cylindrical pump chamber receiving in sliding relationship a manual T-handle assembly for drawing air out of the enlargement receptacle, thereby causing a reduction in air pressure within the enlargement receptacle after a penis has been inserted therein.

The pump of the invention uses a well-established method of enlarging a penis. By repeatedly decreasing the air pressure surrounding the penis, the soft tissues of the penis enlarge, urging blood to flow into the penis, thereby establishing an erection larger than possible using methods unassisted by a pump. It is known that there are both temporary as well as longer-term benefits of this method, since the penis tissues grow in response to the applied low-pressure state, thereby retaining some of the enlargement induced by the lowered pressure, even after the erection subsides.

The invention advances this method and provides advantages over prior art by incorporating important features. For example, the T-shaped handle object improves on prior art by providing a more secure and comfortable grip handle for applying the pumping forces needed to create low air pressure around the penis.

The invention also incorporates the important feature of a substantially right angle between the enlargement receptacle and the pump chamber. The advantage of this feature of the invention is that the pull action required to evacuate the air from the enlargement receptacle can be directed by the user upward, rightward, leftward, or any rotational angle intermediate therewith, rather than directly in line with the penis, as found in the prior art.

Prior art pumps configured so as to facilitate a pump action that pulls the enlargement receptacle away from the base of the penis, thereby disturbing the air-tight seal at the base of the penis, and possibly even risking injury or discomfort. Configurations that incorporate a flexible pipe or tube connecting the enlargement receptacle to the pull action mechanism require that the user to hold the enlargement receptacle in one hand, while pumping with the other hand, resulting in the need to find another means for holding the pump chamber. By incorporating the angled pump chamber within a unified apparatus, a user of the invention can provide a necessary stabilizing balance for the pull action while holding either the enlargement receptacle or the pump chamber, thereby increasing control, and also reducing risk of injury or discomfort. In a general aspect of the invention, an angled male member enlargement pump includes: an enlargement receptacle having an interior sized to accept a male member, the enlargement receptacle also having a first end configured to form a seal when brought into contact with skin of a base of a male member, and a second end. The pump also includes a unidirectional air flow valve having an entry end and an exit end, the entry end being connected to the second end of the enlargement receptacle. The pump further includes an angled pump chamber adapter having a first end and a second end, the first end of the angled pump chamber adapter being connected to the exit end of the unidirectional air flow valve. In addition, the pump includes a pump chamber having a first end and a second end, the first end of the pump chamber being connected with the second end of the angled pump chamber adapter, the second end of the pump chamber having a pump chamber end cap with an opening, the pump chamber further having an air dam shaft that extends through the opening, the air dam shaft having a first end and a second end, the first end of the air dam shaft having a handle, the second end of the air dam shaft being attached to a moveable air dam located within the pump chamber.

In a preferred embodiment, the enlargement receptacle includes a pressure relief assembly. In a further preferred embodiment, the pressure relief assembly includes a self-closing plug for allowing the enlargement receptacle to reach ambient pressure. In an alternate preferred embodiment, the pressure relief assembly includes a stopper shaped so as to allow the enlargement receptacle to reach ambient pressure when a user moves the stopper.

In other preferred embodiments, the enlargement receptacle is transparent.

In still other preferred embodiments, the enlargement receptacle has dimension markings along its length.

In preferred embodiments, the first end of the enlargement receptacle is coated with a material that readily forms a substantially airtight seal with skin.

In yet other preferred embodiments, the angled pump chamber adapter rigidly assumes a 90 degree angle between the enlargement receptacle and the pump chamber.

In preferred embodiments, the angled pump chamber adapter rigidly assumes an angle other than 90 degrees between the enlargement receptacle and the pump chamber.

In other preferred embodiments, the angled pump chamber adapter is adjustable to form a plurality of angles between the enlargement receptacle and the pump chamber.

In further preferred embodiments, the pull handle is substantially T-shaped.

In some preferred embodiments, the pull handle is covered in shock-absorbing material.

In yet other preferred embodiments, the pump chamber is transparent.

In still other preferred embodiments, the enlargement receptacle has a substantially round cross section.

In yet still other preferred embodiments, the pump chamber has a cross sectional area substantially equal to the cross sectional area of the enlargement receptacle.

In another general aspect of the invention, an angled male member enlargement pump includes an enlargement receptacle having an interior sized to accept a male member, the enlargement receptacle also having a first end configured to form a seal when brought into contact with skin of a base of a male member, and a second end. The pump also includes an angled pump chamber adapter having a first end and a second end, the first end of the angled pump chamber adapter being connected to the second end of the enlargement receptacle. The pump further includes a unidirectional air flow valve having an entry end and an exit end, the entry end being connected to the second end of the angled pump chamber. The pump also includes a pump chamber having a first end and a second end, the first end of the pump chamber being connected with the exit end of the unidirectional air flow valve, the second end of the pump chamber having a pump chamber end cap with an opening, the pump chamber further having an air dam shaft that extends through the opening, the air dam shaft having a first end and a second end, the first end of the air dam shaft having a handle, the second end of the air dam shaft being attached to a moveable air dam located within the pump chamber.

In preferred embodiments, the enlargement receptacle is substantially transparent, and includes a pressure relief plug for allowing the enlargement receptacle to reach ambient pressure.

In yet another general aspect of the invention, an angled male member enlargement pump includes an enlargement receptacle for receiving a male member to be enlarged; a unidirectional air flow valve, cooperative with the enlargement receptacle, for permitting air flow in only one direction through the valve; a pump chamber for pulling air along a longitudinal direction of the pump chamber; and an angled pump chamber, cooperative with unidirectional air flow valve and the pump chamber, for establishing and maintaining a substantially 90 degree angle between the one direction through the air flow valve and the longitudinal direction of the pump chamber.

In preferred embodiments, the enlargement receptacle is substantially transparent. In further preferred embodiments, the enlargement receptacle includes a pressure relief plug for allowing the enlargement receptacle to reach ambient pressure.

### BRIEF DESCRIPTION OF THE DRAWING

The invention will be more fully understood by reference to the detailed description, in conjunction with the following figure, wherein:

FIG. 1 is a side view of a preferred embodiment of the pump of the invention showing a pump chamber and an enlargement receptacle, connected by an angled section, the ends being shown simultaneously as perspective views, the pump chamber also including a cut-away view of a section so as to reveal a moveable air dam connected to an air dam shaft.

### DETAILED DESCRIPTION OF THE INVENTION

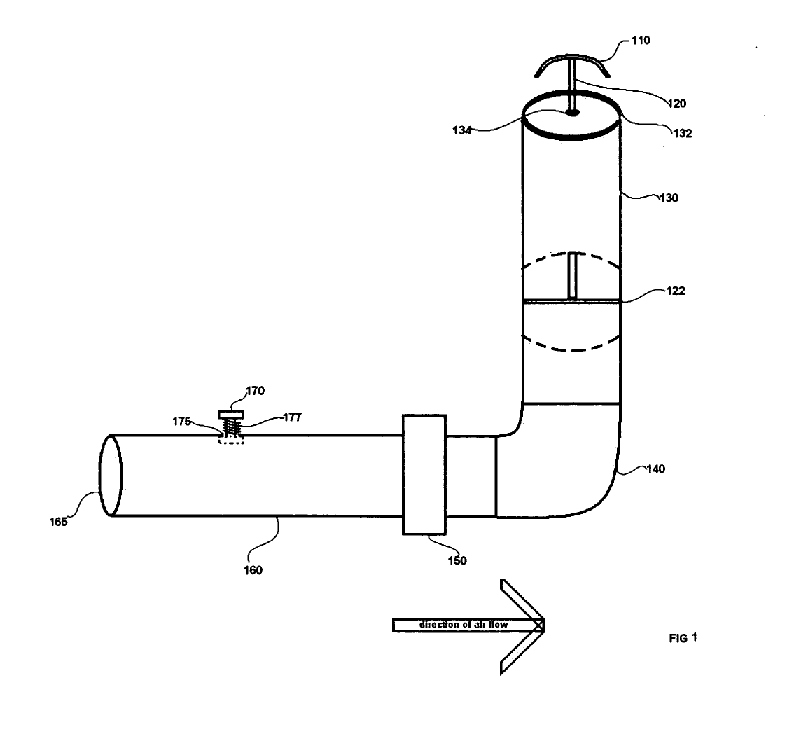

FIG. 1 shows a preferred embodiment of the invention. A pull handle 110 is grasped by a user to move an air dam shaft 120 that is connected to pull handle 110. Air dam shaft 120 is connected to a moveable air dam 122 that sealably and slidably pushes air within the pump chamber 130 towards the pump chamber end cap 132, having an ambient air passageway 134. The air in the pump chamber 130 escapes out the ambient air passageway 134 which is a hole that allows both air and the air dam shaft 120 to pass through.

Moveable air dam 122 also pulls air within pump chamber 130 through an angled pump chamber adapter 140 so as to create low pressure acting upon a unidirectional air flow valve 150. Unidirectional air flow valve 150 allows air to pass from enlargement receptacle 160 into angled pump chamber adapter 140 and pump chamber 130. Taking air out of enlargement receptacle 160 is what creates the low air pressure, sometimes referred to as a “vacuum”, that causes engorgement of the penis, and concomitant enlargement that can become permanent with repeated use of the invention.

Unidirectional air flow valve 150 prevents air from moving from pump chamber 130 and/or angled pump chamber adapter 140 back into enlargement receptacle 160. Thus, when pull handle 110 is pushed in, thereby pushing moveable air dam 122 downward, the air pressure increases so as to push the pressurized air through and/or around moveable air dam 122. The air can then escape through ambient air passageway 134 to commingle with the ambient air.

Enlargement receptacle aperture 165 forms an airtight seal with the base of the penis, so as to help create the low air pressure surrounding the penis. Thus, enlargement receptacle aperture 165 is shaped to be smooth and comfortable. A user can advantageously add a lubricant, such as petrolatum or mineral oil, which will help form an airtight seal between the skin at the base of the penis, and enlargement receptacle aperture 165. To safely and comfortably remove enlargement receptacle 160 from the penis, pressure relief plug 170 allows the user to return the air pressure within enlargement receptacle 160 to ambient pressure.

In a preferred embodiment, the enlargement receptacle 160, angled pump chamber adapter 140, and pump chamber 130 are hollow cylinders. Enlargement receptacle 160 and pump chamber 130 are straight from end-to-end, and angled pump chamber adapter 140 bends substantially 90 degrees from end-to-end. Generally, angled pump chamber adapter 140 can vary from 90 degrees, forming either an acute or obtuse angle, i.e., less than 90 degrees, or more than 90 degrees, but cannot be straight, which would be inconvenient and make the pumping action inefficient, and possibly ineffective.

Enlargement receptacle 160 is preferably made of a clear acrylic or other plastic, and unidirectional air flow valve 150, angled pump chamber adapter 140, and pump chamber 130 are made of opaque acrylic or other plastic. The surfaces of enlargement receptacle aperture 165 are smooth with no sharp edges as it must contact skin at the base of the penis. The interior diameter and length of enlargement receptacle 160 are such that an erect penis does not necessarily contact the interior nor extend beyond the point where enlargement receptacle 160 connects to unidirectional air flow valve 150.

In a preferred embodiment of the invention shown in FIG. 1, enlargement receptacle 160 has an interior diameter of approximately four inches and a length of approximately ten inches, while angled pump chamber adapter 140, and pump chamber 130 have substantially the same interior and exterior diameter as enlargement receptacle 160. Further, in the preferred embodiment, unidirectional air flow valve 150 has substantially the same interior diameter as pump chamber 130, but has a slightly larger exterior diameter to support its function of allowing air to flow in only one direction from enlargement receptacle 160 to angled pump chamber adapter 140.

In the preferred embodiment of the invention in FIG. 1, pump chamber end cap 132 and air dam shaft 120 are made of opaque acrylic or other plastic; pull handle 110 is made from polycarbonate resin, and moveable air dam 122 is made from a flexible material, such as vinyl.

Further in the preferred embodiment of the invention of FIG. 1, pressure relief plug 170 is a spring-actuated, self-closing plug, sealing a relief aperture 175 through which it passes in a side wall of enlargement receptacle 160. Having a shape similar to a spool for storing wire, the central narrow portion of pressure relief plug 170 has a diameter slightly smaller than the relief aperture 175, through which it operates, allowing air to pass around pressure relief plug 170 when it is depressed. A partially compressed relief spring 177 applies pressure between the outer surface of enlargement receptacle 160 and the top of pressure relief plug 170, causing the wide base of pressure relief plug 170 to seal relief aperture 175. The partial compression of the relief spring 177 keeps the wide lower base of pressure relief plug 170 firmly pressing against the inner surface of enlargement receptacle 160 even as the pressure inside enlargement receptacle 160 is reduced. The wide lower base is preferably made of a material that can sealably conform to the inner surface of the enlargement receptacle 160. To relieve the low pressure within the enlargement receptacle 160, an external force is applied to the top of pressure relief plug 170 toward enlargement receptacle 160, further compressing the relief spring 177, moving the wide base of the pressure relief plug 170 away from the inner surface of the enlargement receptacle 160, thereby breaking the air-tight seal and allowing air to flow through relief aperture 175.

An alternate embodiment of the pressure relief plug 170 is a suitably sized rubber stopper that fits in the relief aperture 175. The rubber stopper may also include a flexible lower base that is wider than the relief aperture 175, thereby preventing the rubber stopper from being removed from the relief aperture 175, even when the enlargement receptacle is at ambient room pressure.

An alternative preferred embodiment of the invention includes different versions than shown in FIG. 1, wherein the cross sectional shape of enlargement receptacle 160, unidirectional air flow valve 150, angled pump chamber adapter 140, and pump chamber 130 are of shapes other than round, such as a triangular, square, or octagonal. Further, a plurality of straight and curved segments can be combined to create an alternative cross section of enlargement receptacle 160, unidirectional air flow valve 150, angled pump chamber adapter 140, and pump chamber 130, such that the shape so-formed can meet aesthetic or ergonomic requirements.

In an alternative preferred embodiment of the invention, angled pump chamber adapter 140 can bend flexibly so as to allow for the angle between enlargement receptacle 160 and pump chamber 130 to be changeable by the user to be other than 90 degrees.

Materials other than plastics can be used for each of the parts of the invention such as metal, or wood, or safety-glass. Alternatively many combinations of plastic, wood, safety-glass, and metal can be used for each part, or in combination for any of the parts singly.

The invention is operated manually. A penis is inserted into enlargement receptacle 160 until enlargement receptacle aperture 165 makes contact with the base of the user's penis. The user, or a partner, pulls pull handle 110 slowly, away from pump chamber end cap 132. When pull handle 110 is pulled in this way, moveable air dam 122 forces air from inside pump chamber 130 out through ambient air passageway 134 causing the air pressure in pump chamber 130, angled pump chamber adapter 140, and enlargement receptacle 160 to be reduced below ambient pressure. With unidirectional air flow valve 150 preventing air from flowing back into enlargement receptacle 160 from angled pump chamber adapter 140, and with enlargement receptacle aperture 165 forming a nearly air tight seal with the user's body, the air pressure in enlargement receptacle 160 remains reduced.

After pull handle 110 is pushed back toward pump chamber end cap 132, subsequent pulls of pull handle 110 further reduce the air pressure in enlargement receptacle 160. The reduced air pressure in enlargement receptacle 160 causes the soft tissues of the penis to enlarge, drawing blood into the penis to effect an erection. The penis is removed from the invention after the user depresses pressure relief plug 170, thereby allowing the air pressure inside enlargement receptacle 160 to increase to substantially ambient pressure.

Other modifications and implementations will occur to those skilled in the art without departing from the spirit and the scope of the invention as claimed. Accordingly, the above description is not intended to limit the invention except as indicated in the following claims.

What is claimed is:

1. An angled male member enlargement pump, the pump comprising:
an enlargement receptacle having an interior sized to accept a male member, the enlargement receptacle also having a first end configured to form a seal when brought into contact with skin of a base of a male member, and a second end;
a unidirectional air flow valve having an entry end and an exit end, the entry end being connected to the second end of the enlargement receptacle;
an angled pump chamber adapter having a first end and a second end, the first end of the angled pump chamber adapter being connected to the exit end of the unidirectional air flow valve;
a pump chamber having a first end and a second end, the first end of the pump chamber being connected with the second end of the angled pump chamber adapter, the second end of the pump chamber having a pump chamber end cap with an opening, the pump chamber further having an air dam shaft that extends through the opening, the air dam shaft having a first end and a second end, the first end of the air dam shaft having a handle, the second end of the air dam shaft being attached to a moveable air dam located within the pump chamber.

2. The invention of claim 1, wherein the enlargement receptacle includes a pressure relief assembly.

3. The invention of claim 2, wherein the pressure relief assembly includes:
a self-closing plug for allowing the enlargement receptacle to reach ambient pressure.

4. The invention of claim 2, wherein the pressure relief assembly includes:
a stopper shaped so as to allow the enlargement receptacle to reach ambient pressure when a user moves the stopper.

5. The invention of claim 1, wherein the enlargement receptacle is transparent.

6. The invention of claim 1, wherein the enlargement receptacle has dimension markings along its length.

7. The invention of claim 1, wherein the first end of the enlargement receptacle is coated with a material that readily forms a substantially airtight seal with skin.

8. The invention of claim 1, wherein the angled pump chamber adapter rigidly assumes a 90 degree angle between the enlargement receptacle and the pump chamber.

9. The invention of claim 1, wherein the angled pump chamber adapter rigidly assumes an angle other than 90 degrees between the enlargement receptacle and the pump chamber.

10. The invention of claim 1, wherein the angled pump chamber adapter is adjustable to form a plurality of angles between the enlargement receptacle and the pump chamber.

11. The invention of claim 1, wherein the pull handle is substantially T-shaped.

12. The invention of claim 1, wherein the pull handle is covered in shock-absorbing material.

13. The invention of claim 1, wherein the pump chamber is transparent.

14. The invention of claim 1, wherein the enlargement receptacle has a substantially round cross section.

15. The invention of claim 1, wherein the pump chamber has a cross sectional area substantially equal to the cross sectional area of the enlargement receptacle.

16. An angled male member enlargement pump, the pump comprising:
an enlargement receptacle having an interior sized to accept a male member, the enlargement receptacle also having a first end configured to form a seal when brought into contact with skin of a base of a male member, and a second end;
an angled pump chamber adapter having a first end and a second end, the first end of the angled pump chamber adapter being connected to the second end of the enlargement receptacle;
a unidirectional air flow valve having an entry end and an exit end, the entry end being connected to the second end of the angled pump chamber;
a pump chamber having a first end and a second end, the first end of the pump chamber being connected with the exit end of the unidirectional air flow valve, the second end of the pump chamber having a pump chamber end cap with an opening, the pump chamber further having an air dam shaft that extends through the opening, the air dam shaft having a first end and a second end, the first end of the air dam shaft having a handle, the second end of the air dam shaft being attached to a moveable air dam located within the pump chamber.

17. The angled male member enlargement pump of claim 16, wherein the enlargement receptacle is substantially transparent, and includes a pressure relief plug for allowing the enlargement receptacle to reach ambient pressure.

18. An angled male member enlargement pump, the pump comprising:
an enlargement receptacle for receiving a male member to be enlarged;
a unidirectional air flow valve, cooperative with the enlargement receptacle, for permitting air flow in only one direction through the valve;
a pump chamber for pulling air along a longitudinal direction of the pump chamber; and
an angled pump chamber, cooperative with unidirectional air flow valve and the pump chamber, for establishing and maintaining a substantially 90 degree angle between the one direction through the air flow valve and the longitudinal direction of the pump chamber.

19. The pump of claim 18, wherein the enlargement receptacle is substantially transparent.

20. The pump of claim 18, wherein the enlargement receptacle includes a pressure relief plug for allowing the enlargement receptacle to reach ambient pressure.
